(19) United States
(12) Patent Application Publication
(10) Pub. No.: US 2009/0043357 A1
(43) Pub. Date: Feb. 12, 2009

(54) Wireless real-time feedback control functional electrical stimulation system

(75) Inventors: Kai-yu Tong, Kowloon (HK); Johnson Tin-chak Pang, Kowloon (HK)

Correspondence Address: KAI YU TONG, THE HONG KONG POLYTECHNIC UNIVERSITY, HUNGHOM, KOWLOON, HK

(73) Assignee: The Hong Kong Polytechnic University, Kowloon (HK)

(21) Appl. No.: 11/890,487

(22) Filed: Aug. 7, 2007

(51) Int. Cl. A61N 1/02

(52) U.S. Cl. 607/49

(57) ABSTRACT

The present invention relates methods for assisting a user having paralyzed muscles in walking. The methods focus on addressing foot drop and stimulating arm swing to aid in balance and mimic a “normal” person walk. The methods utilize a system capable of monitoring the gait cycle of the user and wireless transmitting signals to stimulation devices attached to the user's body.

### BACKGROUND

Functional Electrical Stimulation (FES) is a technique for applying electrical currents to neural tissue for the purpose of restoring a degree of control over abnormal or absent body-functions via the generation of muscle contraction. However, accurate and stable control of limbs by FES is difficult because electrically stimulated musculoskeletal systems have strong nonlinearity, time variability, large latency, and fatigue in their response. Moreover, over-stimulated muscles will easily cause fatigue. Optimal control of FES is necessary for extended time use.

For the reasons stated above, as well as other deficiencies in the prior art, a new control system and methods of stimulations have been developed to achieve optimal control of stimulation with a goal of lightening the degree of muscle fatigue.

It is an object of the present system to overcome the disadvantages and problems in the prior art.

### DESCRIPTION

The present inventions proposes a new method of providing electrostimulation to alleviate foot drop and/or initiate arm swing, such method being suitable for optimally controlling stimulation to avoid fatigue over extended time use.

The present invention also proposes new systems for providing electrostimulation, such new system including two separated modules, wherein the modules communicate with one another via wireless means.

The present invention proposes the optimization of electrical stimulation by incorporating the method unto the system of the present invention, as well as by positioning components of the system in strategic places on the user's body.

The present invention proposes the method and system to produce artificial arm swing through the stimulation of triceps to generate the elbow extension for body balancing to improve gait quality. Further, the method will address foot drop to make walking easier and more “normal”-like.

These and other features, aspects, and advantages of the apparatus and methods of the present invention will become better understood from the following description, appended claims, and accompanying drawings where:

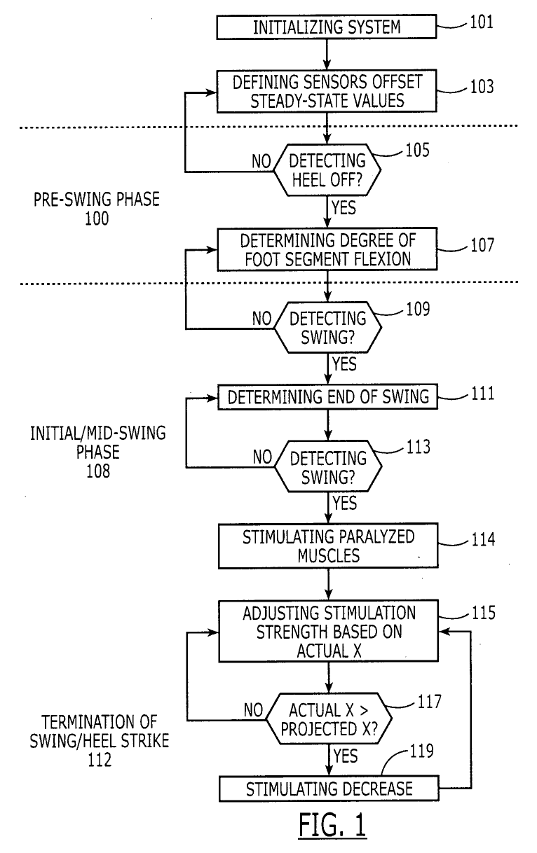

FIG. 1 shows a method of stimulating paralyzed muscles of a user for assisting in walking.

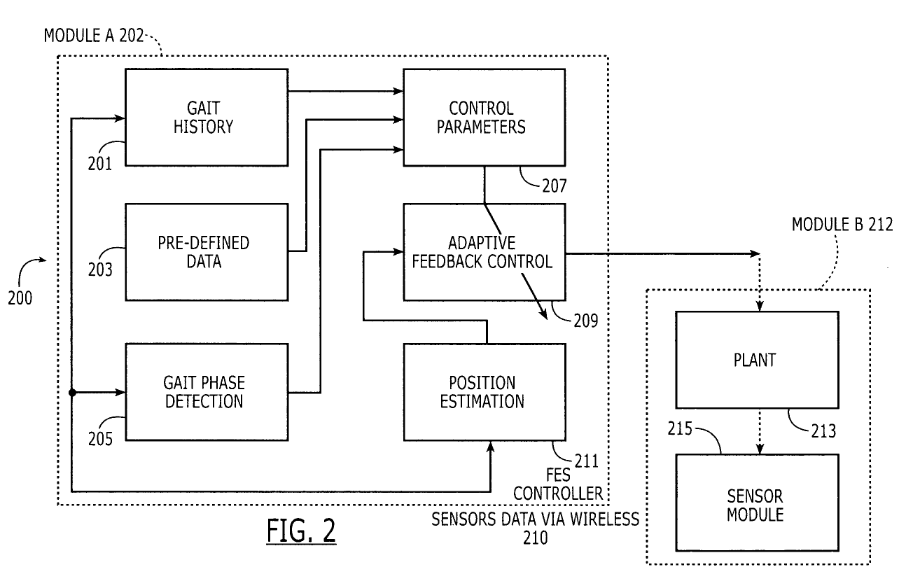

FIG. 2 shows the system of the invention, containing a module A and module B.

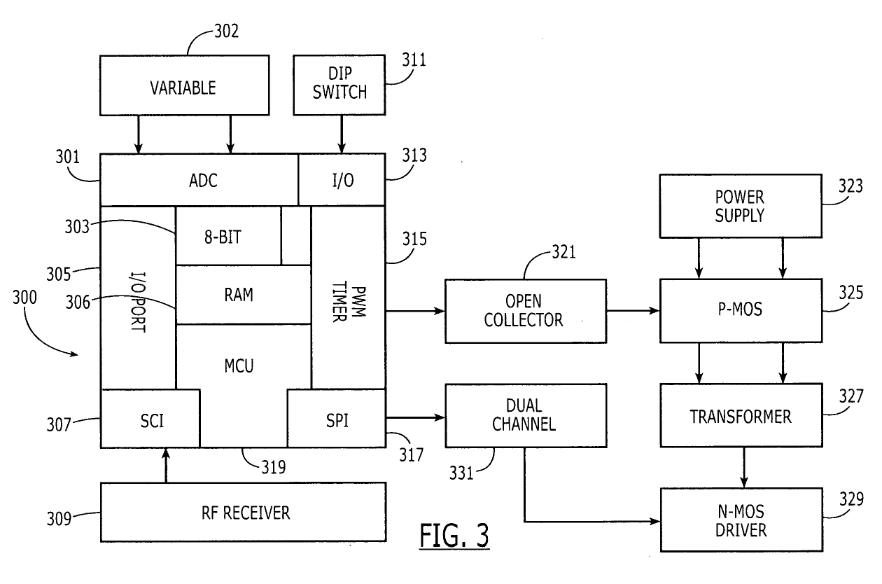

FIG. 3 shows an embodiment for module B of the system.

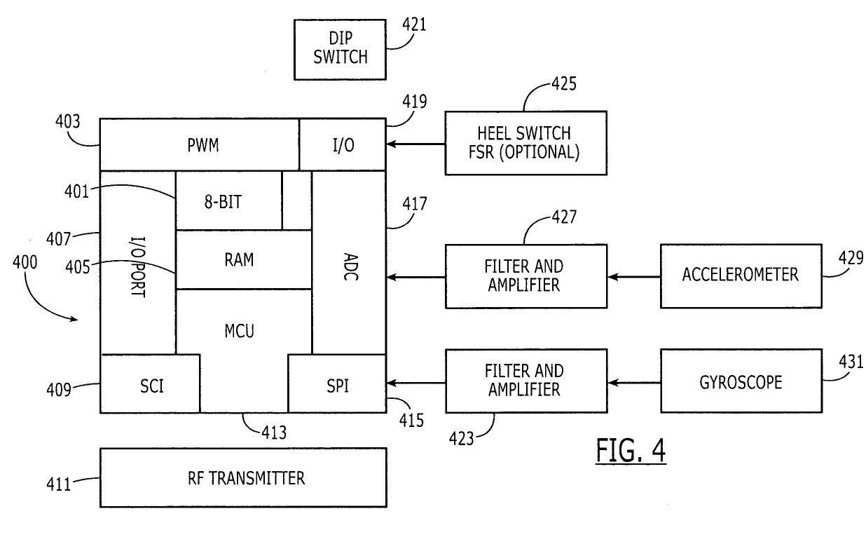

FIG. 4 shows an embodiment for module A of the system.

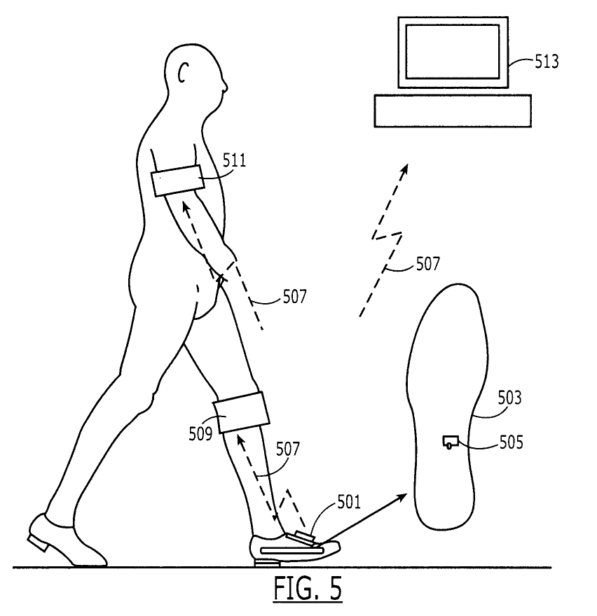

FIG. 5 shows a user employing the system of the invention.

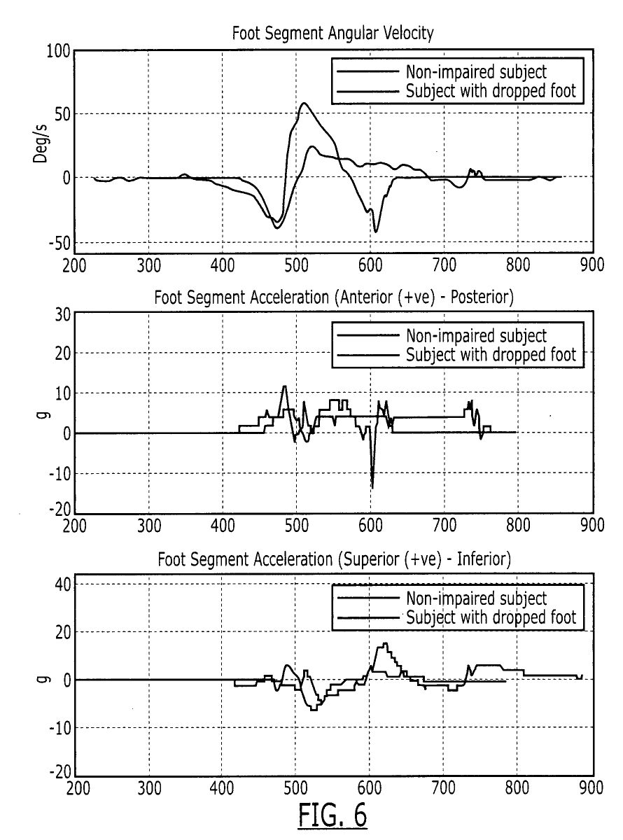

FIG. 6 shows signals generated by the systems.

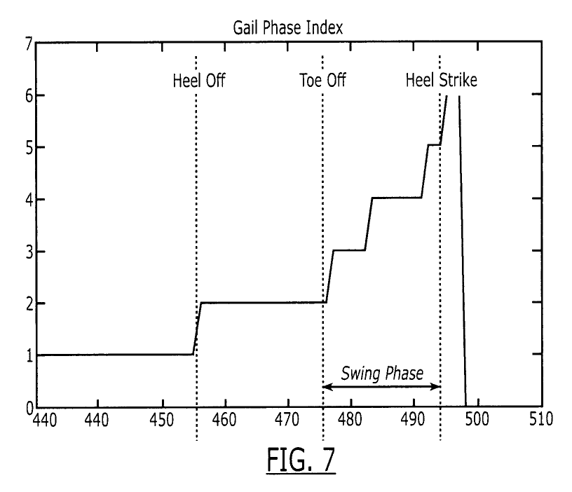

FIG. 7 shows examples of the gait phase.

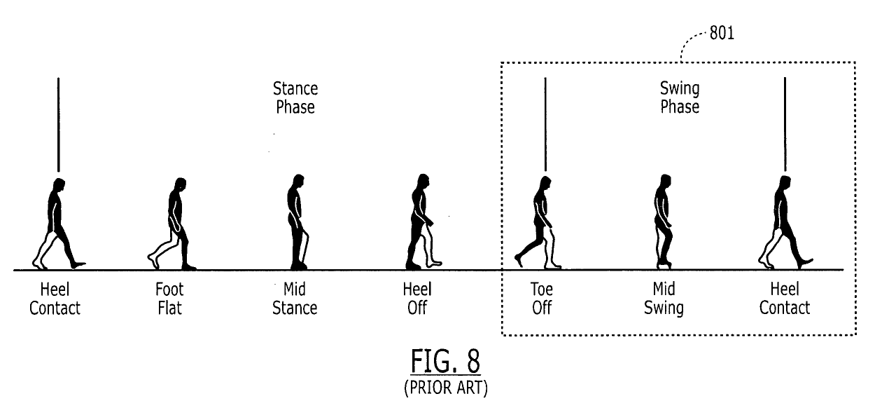

FIG. 8 shows prior art gait cycle.

The following description of certain exemplary embodiment(s) is merely exemplary in nature and is in no way intended to limit the invention, its application, or uses. Throughout this description, the term “gait” refers to a particular way or manner of moving on foot.

Now, specifically to FIGS. 1-8, The present invention has as a goal addressing paralyzed muscles for walking functions, such paralysis being brought about by stroke or lesions. Addressing the paralyzed muscles can occur by stimulating the muscles during the swing phase of a gait cycle, notably to reduce or positively influence foot drop and enhance arm swing. The present invention includes methods of stimulating arm swing and decreasing foot drop, and apparatuses incorporating wireless means for stimulating paralyzed muscles. It is believed the present invention will assist people suffering from debilitating illnesses by electronically stimulating nerve and muscles to improve walking gesture.

FIG. 1 is a method of assisting people in walking, such people generally having paralyzed muscles, utilizing a stimulation apparatus of the present invention. The method can be broken down into 3 sections in the swing phase of a gait cycle: pre-swing phase 100, initial to mid-swing phase 108, and termination of the swing to the heel strike phase 112.

Firstly, in the pre-swing phase 100 the system delivering the method is initialized 101. Initialization can include turning on the instrument, allowing the system to perform a systems check, having the system establish remote contacts, and the like.

The system can then define required values necessary to successfully provide the method 103. Values can refer to angular values, timing values, etc. As the system will rely on on-board instrumentation for determining when the user is in a particular phase of the gait, the instruments should be “zeroed” to offset the initial values to avoid false positives or missed periods of needed stimulation for the user. The instrumentation may also be modified to provide training and conditioning, for example decreasing or increasing the sensitivity of the instrumentation to provide a less stringent or more stringent exercise.

The algorithmic function of detecting whether the user's heel is “off” will then be determined 105. As is well known in the art, the pre-swing phase initiates when the heel of a foot is lifted off of the surface while the toes remain in contact with the surface (see FIG. 8 for more understanding). If the heel is not detected “off”, the algorithm is looped back to defining values 103. In the event the heel is detected “off”, further analysis is made.

If the heel is detected “off”, a determination of the degree of the foot segment flexion is made 107. As is known in the art, in the period between heel off and toe off, the ankle moves from dorsiflexion to plantarflexion. Generally, from the time of heel-off, the foot measures at an angle of from about 20 to about 35 degree from the surface. The increase in ankle degree occurs as the swing progresses, and measurement is continually made.

As the swing progresses to the toe-off, an algorithm is enacted to determine whether swing is detected 109. A swing can be detected by noting the increase in angle change of the foot to the surface. In another embodiment, a detection of swing can be determined by noting the changes in the hips, knees, ankle, or upper body. Changes that can be noted include angle change, pressure, weight, flexion and/or extension. If swing is not detected, the algorithm is looped for determination of the foot segment flexion degree.

If the swing is detected, a determination is made when the swing has ended 111. As is well known in the art, a swing is generally terminated at the heel strike of the swinging foot. Determining the end of the swing can be based on decrease angle between the foot and the contact surface. In another embodiment, determination of the end of the swing can be based on change of pressure, angle, weight, flexion and/or extension as exhibited by the hips, knees, ankle, or upper body.

As is well known, an end of a swing on one foot will initiate the beginning of the swing phase on the alternate foot.

An algorithm is then made as to whether the alternate foot is being swung 113. If “yes”, stimulating paralyzed muscles can occur 114. Stimulation of paralyzed muscles is electrical-stimulation, and can occur by positioning electrical conducting devices on or around the paralyzed muscles of the user. The electrical conducting devices can be pads, wrap around implements, wires, or embedded devices into the body of the user. Stimulation preferably occurs at the proper moment in order to improve the walking pattern to be appeared more “normal”, i.e. similar to a non-paralyzed person's walk.

Stimulation may occur to the lower limb of the user, such as leg, or an upper limb such as the arm. For example, stimulation is focused on the arm of the user to mimic the arm swing of a “normal” person. In one embodiment, more than one body part of a person is stimulated, for example both arm and leg are stimulated. In such an embodiment, stimulation of a lower limb and upper limb may alternate to simulate a “normal person” and better effective walking. As will be discussed later, stimulation will occur via wireless means.

Stimulation may be adjusted as need be to better effectuate a particular gait 115. For example, if a gait is at a higher rate of speed than normal speed of walking, strong stimulation may be required to be delivered to the user to provide faster muscle contraction and wider range of movement on the paralyzed muscle. In another example, the stimulation may be decreased if a slower gait is being exhibited. Adjustment of the stimulation will be based on the actual variable compared against a projected variable 117. For example the actual speed of the user can be compared against a projected, or programmed, speed. If necessary, stimulation can be decreased 119. In other embodiments, stimulation may be increased.

FIG. 2 is an embodiment of a system 200 of the present invention used for assisting a user in walking, such user having paralyzed muscles. The system 200 includes two modules, a module A 202 and a module B 212. Module A 202 serves as a monitoring and data transmitting device, and Module B 212 serves as a data processing and stimulation adjuster for paralyzed muscles.

Module A 202 can include a memory device for storing gait history 201, a device for storing predefined data 203, a gait phase detector 205, a controller 207, a feedback controller 209, and a device for estimating the position of the user 211. Module A 202 also includes sensors for passing data via wireless means 210.

Module B 212 can include a plant device 213 for attachment to a muscle location on the user, and a sensor module 215 for detecting a wireless signal sent from Module A 202.

Module A 202 and Module B 212 can be configured to operate in 3 modes. Mode 1 is a foot drop stimulator with real-time feedback control. In this mode, Module A is attached to a foot segment to act as an electro-stimulation trigger and foot flexion monitoring device. Data captured by the sensors are transmitted via wireless means, such as radio-frequency, to Module B. Module B processes the received data with the gait phase detection algorithm of the present invention and applies a different control strategy to adjust stimulation parameters at different gait phases.

Mode 2 is a combination mode, combining Modules A and B into a single device to act as a foot drop stimulator. The single device is attached to the shank and electro-stimulation is trigger by the movement of the device.

Mode 3 is a combination of modes 1 and 2, i.e., a foot drop stimulator combined with an arm swing stimulator. In this mode, Module A monitors the gait phase of the user and transmits gait data to module B of the foot drop stimulator and arm swing stimulator. The arm swing stimulator produces stimulation at appropriate gait phase, and the shank stimulator addresses the foot drop problem.

The transmission of data between Module A and Module B occurs by wireless means, for example RF, WIFI, satellites, BLUETOOTH®, and other wireless technologies. Preferably, a wireless transmitter is installed in Module A and a receiver is installed in Module B.

FIG. 3 is an embodiment of Module B 300 of the system of the present invention. The embodiment 300 is primarily composed of a main control unit (MCU) 310 such as a microprocessor, and radio frequency receiver 309. The embodiment 300 further includes resistors 302, switches 311, converters 301, memory such as RAM 306, pulse with modulators 315, input/output parts 313/305, interfaces (SPI and SCI) 317/307, transistors 325/329, transformer 327, power supply 323, and connection means 321/331. It will be well known to one with ordinary skill in the art that the components may be substituted or supplemented with other electronic components.

FIG. 4 is an embodiment of Module A of the system of the present invention. The embodiment 400 includes filters and amplifiers 423/427, main control units 413 such as microprocessors, and a radio frequency transmitter. Notably, the embodiment 400 includes an accelerometer 429 and gyroscope 431 as gait phase detectors, allowing the system to determine at what phase of the gait cycle the user is in. This allows the apparatus to time when a stimulation signal should be sent from Module A to Module B. The embodiment 400 can also include memory such as RAM 405, input/output parts 407/419, pulse width modulators 403, interfaces 409/415, converters 417, and switches 421. In the event the system is used for a foot drop stimulator, the embodiment 400 can include a heel switch 425. It will be known to one with ordinary skill in the art that the components may be substituted or supplemented with other electronic components.

FIG. 5 exhibits the attachment of the system on a user, the system comprising a Module A 501 and Modules B 511/509, wherein the Modules B are an arm mounted module B 511 and a shank mounted module B 511.

Module A 501 is preferably attached adjacent to the foot of the user. In one embodiment, the Module A 501 can be attached to the shoe of the user, in one embodiment on the top side of the shoe. The gait phase detector 505 may be incorporated into the inside sole 503 of the shoe. As previously mentioned, the gait phase detector 505 is used to determine whether the foot is in the swing phase of the gait cycle and, if so, what section of the swing phase.

In use, the Module A 501 transmits signals 507 to the modules B 511/509, pertaining to whether electro-stimulation by the Module B should be provided. The Module A 501 may also send a signal to a remote location 513, such as a computer system, for recording information about the user's gait, swing phase, and the like. In a preferred embodiment, if mode 3 is enacted, i.e. a Module B is utilized for arm swing and foot drop stimulation the modules should either be positioned in an alternating manner (left leg/right arm, or right leg/left arm) or stimulation should be provided in an alternating manner, i.e., left leg stimulation, no right arm stimulation/no left leg stimulation, right arm stimulation. In this way, the gait cycle of a “normal” person can better be mimiced by the muscle-paralyzed user.

The system will operate according to the previously mentioned method (FIG. 1) including detecting heel off, determining degree, detecting swing, stimulating muscles, and adjusting stimulation as needed.

### EXAMPLES

FIG. 6 shows the signals as captured by the gait phase detectors gyroscope and accelerometer during the gait cycle for normal and stroke subject with foot drop problem.

FIG. 7 shows examples of the gait phase detected by the system of the present invention utilizing the gait phase detection method. The gait phases in one gait cycle could be identified and given a gait phase index.

FIG. 8 shows the gait cycle for a human. The system and method of the instant invention have their usefuless during the swing phase 801 of the cycle

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A method of assisting a user with paralyzed muscles in walking, comprising the steps of:
initializing a functional electrical stimulation system;
defining sensor offset and steady-state values;
detecting a heel “off”
If NOT, then
redefining sensors offset and steady state values
otherwise,
determining the degree of foot segment flexion;
detecting a swing
If NOT, then
redetermining the degree of foot segment flexion;
otherwise,
stimulating paralyzed muscles;
adjusting stimulation strength based on an actual measurement;
determining whether the actual measurement is greater then the projected measurement
If yes, then
decreasing stimulation.

2. The method of claim 1, wherein determining the degree of foot segment flexion measures from about 20° to about 35° from the surface.

3. The method of claim 1, wherein detection of a swing can be determined by noting the increase in angle change of the foot to the surface.

4. The method of claim 1, wherein determining the end of the swing can be based on a decrease angle between the foot and the contact surface.

5. The method of claim 1, wherein stimulating the paralyzed muscles occurs by electrical stimulation.

6. A method for addressing foot drop of a user, comprising the steps of
attaching a first module for transmitting a signal to a foot segment of said user;
attaching a second module for providing stimulation to a shank of said user; and
sending a signal wirelessly from said first module to said second module in response to movement of said first module.

7. The method for addressing foot drop in claim 6, wherein said first module includes a memory storage device, one or more gait phase detector, a controller, a feedback controller, a device for estimating the position of the user, are wireless data transmitter.

8. The method for addressing foot drop in claim 6, wherein said second module includes a plant device for attachment to said shank and a sensor for detecting a wireless signal.

9. The method for addressing foot drop in claim 7, wherein said gait phase detectors can be an accelerometer and a gyroscope.

10. The method for addressing foot drop in claim 6, wherein sending said signal wirelessly occurs by radio-frequency.

11. The method for addressing foot drop in claim 6, wherein movement of the first module occurs during a gait phase cycle.

12. A method for addressing foot drop and stimulating arm swing of a muscle paralyzed user, comprising the steps of
attaching a first module for transmitting a signal to a foot segment of said user;
attaching second modules for providing stimulation to a shank and the arm of said user; and
sending a signal wirelessly said first module to said second modules in response to the gait phase cycle of the user.
